(19) United States
(12) Patent Application Publication
(10) Pub. No.: US 2007/0191248 A1
(43) Pub. Date: Aug. 16, 2007

(54) Detergent compositions

(76) Inventors: Philip Frank Souter, Northumberland (GB); John Allen Burdis, Newcastle Upon Tyne (GB); Kim Borch, Davis, CA (US); Allan Svendsen, Horsholm (DK); Mikael Mikkelsen, Smorum (DK); Jesper Vind, Vaerlose (DK); Neil Joseph Lant, Newcastle upon Tyne (GB)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/656,261

(22) Filed: Jan. 22, 2007

Related U.S. Application Data
(60) Provisional application No. 60/761,108, filed on Jan. 23, 2006.
(60) Provisional application No. 60/796,325, filed on Apr. 28, 2006.
(60) Provisional application No. 60/854,845, filed on Oct. 27, 2006.

(51) Int. Cl. C11D 3/386; C12N 9/20

(52) U.S. Cl. 510/320; 435/198

(57) ABSTRACT

The present invention relates to detergent compositions comprising a detergent ingredient and a specific lipase variant with reduced potential for odor generation and a good relative performance versus the parent lipase.

### CROSS-REFERENCES TO RELATED APPLICATIONS

This application claims priority under 35 U.S.C. § 119(e) to U.S. Provisional Application Ser. No. 60/761,108 filed Jan. 23, 2006, U.S. Provisional Application Ser. No. 60/796,325 filed Apr. 28, 2006, and U.S. Provisional Application Ser. No. 60/854,845 filed Oct. 27, 2006.

### FIELD OF THE INVENTION

The present invention relates to detergent compositions, particularly laundry detergents, comprising lipolytic enzymes.

### BACKGROUND OF THE INVENTION

Improved removal of greasy soils is a constant aim for detergent manufacturers, especially in the laundry context. In spite of the use of many effective surfactants and combinations of surfactants, especially when used at low water temperatures, many surfactant-based products still do not achieve complete removal of greasy/oily soils. Lipase enzymes have been used in detergents since the late 1980s for removal of fatty soils by breakdown of fatty soils into tri-glycerides.

Until relatively recently, the main commercially available lipase enzymes, such as Lipolase (trade name, Novozymes) worked particularly effectively at the lower moisture levels of the drying phase of the wash process. These enzymes tended to produce significant cleaning only in the second wash step with fat breakdown significant only on soils remaining on laundered clothes during the drying stage, the broken down fats then being removed in the next washing step. However, more recently, higher efficiency lipases have been developed that also work effectively during the wash phase of the cleaning process, so that as well as cleaning in the second washing step, a significant improvement in cleaning effect due to lipase enzyme can be found in the first wash-cycle. Examples of such enzymes are as described in U.S. Pat. No. 6,939,702B1, WO00/60063 and Research Disclosure IP6553D. Such enzymes are referred to below as first wash lipases.

In addition, consumers prefer that articles, such as garments, be as clean as possible. Such consumers typically associate the odor of a cleaned or treated article with the degree of cleanliness of such article. Thus, the effectiveness of a cleaning and/or treatment composition, from a consumer's perspective, is typically directly linked with the odor that such composition imparts to an article that is cleaned or treated with such composition. Applicants recognized that certain materials, such as esterases and lipases, can generate objectionable fatty acid odors, particularly short-chain fatty acid odors such as the odor of butyric acid. However, such materials can be particularly effective cleaning agents. Unfortunately, consumers typically associate the odors resulting from the use of such agents with a lack of cleanliness. Examples of reduced odour variants with a C-terminal extension are shared in WO02/062973, but these lipase variants do not demonstrate the strong wash performance of the first wash lipases such as those from WO00/60063 including the variant sold under the tradename Lipex®.

Thus, there remains a need for a detergent compositions comprising lipolytic enzymes for excellent greasy/oily soils removal while not generating any objectionable fatty acid odors.

### SUMMARY OF THE INVENTION

The present invention relates to detergent compositions comprising a detergent ingredient and a lipase variant having an average Relative performance (RPavg) of at least 0.8 and a Benefit-Risk (BR) of at least 1.1 at the test conditions given in the specification.

### SEQUENCE LISTING

SEQ ID NO: 1 shows the DNA sequence encoding lipase from Thermomyces lanoginosus.

SEQ ID NO: 2 shows the amino acid sequence of a lipase from Thermomyces lanoginosus.

SEQ ID NO: 3 and SEQ ID NO: 4 show sequences used for alignment example.

### DETAILED DESCRIPTION OF THE INVENTION

Definitions

Lipase activity: The term “lipase activity” is defined herein as a carboxylic ester hydrolase activity which catalyzes the hydrolysis of triacylglycerol under the formation of diacylglycerol and a carboxylate. For purposes of the present invention, lipase activity is determined according to the procedure described in “Lipase activity” in “Materials and Methods”. One unit of lipase activity is defined as the amount of enzyme capable of releasing 1.0 micro mole of butyric acid per minute at 30° C., pH 7.

The polypeptides of the present invention have at least 70%, such at least 75% or 80% or 85% or 90%, more preferably at least 95%, even more preferably 96% or 97%, most preferably 98% or 99%, and even most preferably at least 100% of the lipase activity measured as Relative Performance of the polypeptide consisting of the amino acid sequence shown as the mature polypeptide of SEQ ID NO:2, with the substitutions T231R+N233R.

Isolated polypeptide: The term “isolated polypeptide” as used herein refers to a polypeptide which is at least 20% pure, preferably at least 40% pure, more preferably at least 60% pure, even more preferably at least 80% pure, most preferably at least 90% pure, and even most preferably at least 95% pure, as determined by SDS-PAGE.

Substantially pure polypeptide: The term “substantially pure polypeptide” denotes herein a polypeptide preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of other polypeptide material with which it is natively associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation.

The polypeptides of the present invention are preferably in a substantially pure form. In particular, it is preferred that the polypeptides are in “essentially pure form”, i.e., that the polypeptide preparation is essentially free of other polypeptide material with which it is natively associated. This can be accomplished, for example, by preparing the polypeptide by means of well-known recombinant methods or by classical purification methods.

Herein, the term “substantially pure polypeptide” is synonymous with the terms “isolated polypeptide” and “polypeptide in isolated form.”

Identity: The relatedness between two amino acid sequences or between two nucleotide sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”; e.g. amino acids 1 to 269 of SEQ ID NO:2) and a different amino acid sequence (“foreign sequence”) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap (in the alignment example below this is represented by “|”). The length of a sequence is the number of amino acid residues in the sequence (e.g. the length of SEQ ID NO:2 is 269).

In the alignment example below, the overlap is the amino acid sequence “HTWGER-NL” of Sequence A; or the amino acid sequence “HGWGEDANL” of Sequence B. In the example a gap is indicated by a “-”.

### Alignment Example

| | |
|---|---|
| Sequence A: | ACMSHTWGER-NL |
| | \| \|\|\| \|\| |
| Sequence B: | HGWGEDANLAMNPS |

Polypeptide Fragment: The term “polypeptide fragment” is defined herein as a polypeptide having one or more amino acids deleted from the amino and/or carboxyl terminus of SEQ ID NO: 2 or a homologous sequence thereof, wherein the fragment has lipase activity.

Subsequence: The term “subsequence” is defined herein as a nucleotide sequence having one or more nucleotides deleted from the 5′ and/or 3′ end of SEQ ID NO: 1 or a homologous sequence thereof, wherein the subsequence encodes a polypeptide fragment having lipase activity.

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form. In particular, it is preferred that the polynucleotides disclosed herein are in “essentially pure form”, i.e., that the polynucleotide preparation is essentially free of other polynucleotide material with which it is natively associated. Herein, the term “substantially pure polynucleotide” is synonymous with the terms “isolated polynucleotide” and “polynucleotide in isolated form.” The polynucleotides may be of genomic, cDNA, RNA, semisynthetic, synthetic origin, or any combinations thereof.

cDNA: The term “cDNA” is defined herein as a DNA molecule which can be prepared by reverse transcription from a mature, spliced, mRNA molecule obtained from a eukaryotic cell. cDNA lacks intron sequences that are usually present in the corresponding genomic DNA. The initial, primary RNA transcript is a precursor to mRNA which is processed through a series of steps before appearing as mature spliced mRNA. These steps include the removal of intron sequences by a process called splicing. cDNA derived from mRNA lacks, therefore, any intron sequences.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polynucleotide encoding a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG. The coding sequence may a DNA, cDNA, or recombinant nucleotide sequence.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the invention, and which is operably linked to additional nucleotides that provide for its expression.

Host cell: The term “host cell”, as used herein, includes any cell type which is susceptible to transformation, transfection, transduction, and the like with a nucleic acid construct comprising a polynucleotide of the present invention.

Modification: The term “modification” means herein any chemical modification of the polypeptide consisting of the mature polypeptide of SEQ ID NO: 2 as well as genetic manipulation of the DNA encoding that polypeptide. The modification(s) can be substitution(s), deletion(s) and/or insertions(s) of the amino acid(s) as well as replacement(s) of amino acid side chain(s).

Artificial variant: When used herein, the term “artificial variant” means a polypeptide having lipase activity produced by an organism expressing a modified nucleotide sequence of SEQ ID NO: 1. The modified nucleotide sequence is obtained through human intervention by modification of the nucleotide sequence disclosed in SEQ ID NO: 1.

Relative performance (RP): The term relative performance reflects performance of the enzyme variant compared to a reference enzyme when measured as the brightness of the color of the textile samples washed with that specific enzyme variant as described in Example 2 of the present specification.

Risk (R): The term “risk” and risk factor are used interchangeably in the present specification and is the ratio between the amount of released butyric acid from the lipase variant washed swatch and the amount of released butyric acid from a swatch washed with the mature part of the lipase of SEQ ID NO: 2, after both values have been corrected for the amount of released butyric acid from a non-lipase washed swatch.

Benefit-Risk factor (BR): The Benefit-Risk factor describes the wash performance compared to risk for odor. Thus BR=RP_{avg}/R.

Conventions for Designation of Variants:

In describing lipase variants according to the invention, the following nomenclature is used for ease of reference: Original amino acid(s):position(s):substituted amino acid(s)

According to this nomenclature, for instance the substitution of glutamic acid for glycine in position 195 is shown as G195E. A deletion of glycine in the same position is shown as G195*, and insertion of an additional amino acid residue such as lysine is shown as G195GK.

Where a specific lipase contains a “deletion” in comparison with other lipases and an insertion is made in such a position this is indicated as *36D for insertion of an aspartic acid in position 36.

Multiple mutations are separated by pluses, i.e.: R170Y+G195E, representing mutations in positions 170 and 195 substituting tyrosine and glutamic acid for arginine and glycine, respectively.

X231 indicates the amino acid in a parent polypeptide corresponding to position 231, when applying the described alignment procedure. X231R indicates that the amino acid is replaced with R. For SEQ ID NO:2 X is T, and T231R thus indicates a substitution of T in position 231 with R. Where the amino acid in a position (e.g. 231) may be substituted by another amino acid selected from a group of amino acids, e.g. the group consisting of R and P and Y, this will be indicated by X231R/P/Y.

In all cases, the accepted IUPAC single letter or triple letter amino acid abbreviation is employed.

### DETAILED DESCRIPTION OF THE INVENTION

Polypeptides Having Lipase Activity

The isolated polypeptides having a lipase activity, of the present invention, are selected from the group consisting of lipases having a RP of at least 0.8 and a BR of at least 1.1 at the test conditions given in the specification.

In a preferred embodiment the lipase has a RP of at least 0.9, such as 1.0 or 1.1. In an even more preferred embodiment the lipase has a RP of at least 1.2, such as 1.3 or even 1.4.

In another preferred embodiment the lipase has a BR of at least 1.2, such as 1.3 or even 1.4. In an even more preferred embodiment the lipase has a BR of at least 1.5, such as 1.6 or even 1.7.

In a further aspect the polypeptide of the present invention further has a relative LU/A280 less than 1, such as less than 0.95 at the test conditions given in the specification. In a preferred embodiment the relative LU/A280 is less than 0.90, such as less than 0.85 or even less than 0.80.

In a further aspect, the isolated polypeptides of the present invention, have an amino acid sequence which is comprised by or comprises SEQ ID NO:2, or an allelic variant thereof, and which further has BR of at least 1.1 and RP of at least 0.8. In another aspect, the isolated polypeptides of the present invention, have an amino acid sequence which is comprised by or comprises the mature part of SEQ ID NO:2, or an allelic variant thereof, and which further has BR of at least 1.1 and RP of at least 0.8

In a still further aspect, the isolated polypeptides of the present invention, have an amino acid sequence which has a degree of identity to the mature polypeptide of SEQ ID NO: 2 (i.e., the mature polypeptide) of at least 80%, such as at least 85% or 90%, or at least 95%, preferably at least 97%, most preferably at least 98%, and even most preferably at least 99%, which have lipase activity (hereinafter “homologous polypeptides”). In a preferred aspect, the homologous polypeptides have an amino acid sequence which differs by ten amino acids, preferably by five amino acids, more preferably by four amino acids, even more preferably by three amino acids, most preferably by two amino acids, and even most preferably by one amino acid from the mature polypeptide of SEQ ID NO: 2.

In a further aspect, the isolated polypeptides having lipase activity of the present invention, are encoded by polynucleotides which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 644 to 732 of SEQ ID NO: 1, (ii) the cDNA sequence contained in nucleotides 644 to 732 of SEQ ID NO: 1, (iii) a subsequence of (i) or (ii), or (iv) a complementary strand of (i), (ii), or (iii) (J. Sambrook, E. F. Fritsch, and T. Maniatus, 1989, Molecular Cloning, A Laboratory Manual, 2d edition, Cold Spring Harbor, N.Y.). A subsequence of SEQ ID NO: 1 contains at least 100 contiguous nucleotides or preferably at least 200 contiguous nucleotides. Moreover, the subsequence may encode a polypeptide fragment which has lipase activity.

The nucleotide sequence of SEQ ID NO: 1 or a subsequence thereof, as well as the amino acid sequence of SEQ ID NO: 2 or a fragment thereof, may be used to design a nucleic acid probe to identify and clone DNA encoding polypeptides having lipase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes which are at least 600 nucleotides, at least preferably at least 700 nucleotides, or more preferably at least 800 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may, therefore, be screened for DNA which hybridizes with the probes described above and which encodes a polypeptide having lipase activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO: 1 or a subsequence thereof, the carrier material is used in a Southern blot.

For purposes of the present invention, hybridization indicates that the nucleotide sequence hybridizes to a labeled nucleic acid probe corresponding to the nucleotide sequence shown in SEQ ID NO: 1, its complementary strand, or a subsequence thereof, under very low to very high stringency conditions. Molecules to which the nucleic acid probe hybridizes under these conditions can be detected using X-ray film.

In a still further aspect, the variant of the present invention can be artificial variants comprising a conservative substitution, deletion, and/or insertion of one or more amino acids of SEQ ID NO: 2 or the mature polypeptide thereof, said variants having a BR of at least 1.1 and a RP of at least 0.8. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/le, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., lipase activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. (Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

In one aspect, the total number of amino acid substitutions, deletions and/or insertions of amino acids 1 to 291 of SEQ ID NO: 2 is 10, preferably 9, more preferably 8, more preferably 7, more preferably at most 6, more preferably at most 5, more preferably 4, even more preferably 3, most preferably 2, and even most preferably 1.

Identification of Regions and Substitutions.

The positions referred to in Region I through Region IV below are the positions of the amino acid residues in SEQ ID NO:2. To find the corresponding (or homologous) positions in a different lipase, the procedure described in “Homology and alignment” is used.

Substitutions in Region I

Region I consists of amino acid residues surrounding the N-terminal residue E1. In this region it is preferred to substitute an amino acid of the parent lipase with a more positive amino acid. Amino acid residues corresponding to the following positions are comprised by Region I: 1 to 11 and 223-239. The following positions are of particular interest: 1, 2, 4, 8, 11, 223, 227, 229, 231, 233, 234 and 236. In particular the following substitutions have been identified: X1N/*, X4V, X227G, X231R and X233R.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

Substitutions in Region II

Region II consists of amino acid residues in contact with substrate on one side of the acyl chain and one side of the alcohol part. In this region it is preferred to substitute an amino acid of the parent lipase with a more positive amino acid or with a less hydrophobic amino acid. Amino acid residues corresponding to the following positions are comprised by Region II: 202 to 211 and 249 to 269. The following positions are of particular interest: 202, 210, 211, 253, 254, 255, 256, 259. In particular the following substitutions have been identified: X202G, X210K/W/A, X255Y/V/A, X256K/R and X259G/M/Q/V.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

Substitutions in Region III

Region III consists of amino acid residues that form a flexible structure and thus allowing the substrate to get into the active site. In this region it is preferred to substitute an amino acid of the parent lipase with a more positive amino acid or a less hydrophobic amino acid. Amino acid residues corresponding to the following positions are comprised by Region III: 82 to 102. The following positions are of particular interest: 83, 86, 87, 90, 91, 95, 96, 99. In particular the following substitutions have been identified: X83T, X86V and X90A/R.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

Substitutions in Region IV

Region IV consists of amino acid residues that bind electrostatically to a surface. In this region it is preferred to substitute an amino acid of the parent lipase with a more positive amino acid. Amino acid residues corresponding to the following positions are comprised by Region IV: 27 and 54 to 62. The following positions are of particular interest: 27, 56, 57, 58, 60. In particular the following substitutions have been identified: X27R, X58N/AG/T/P and X60V/S/G/N/R/K/A/L.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

Amino Acids at Other Positions

The parent lipase may optionally comprise substitutions of other amino acids, particularly less than 10 or less than 5 such substitutions. Examples are substitutions corresponding to one or more of the positions 24, 37, 38, 46, 74, 81, 83, 115, 127, 131, 137, 143, 147, 150, 199, 200, 203, 206, 211, 263, 264, 265, 267 and 269 of the parent lipase. In a particular embodiment there is a substitution in at least one of the positions corresponding to position 81, 143, 147, 150 and 249. In a preferred embodiment the at least one substitution is selected from the group consisting of X81Q/E, X143S/C/N/D/A, X147M/Y, X150G/K and X249R/I/L.

The variant may comprise substitutions outside the defined Regions I to IV, the number of substitutions outside of the defined Regions I to IV is preferably less than six, or less than five, or less than four, or less than three, or less than two, such as five, or four, or three, or two or one. Alternatively, the variant does not comprise any substitution outside of the defined Regions I to IV.

Further substitutions may, e.g., be made according to principles known in the art, e.g. substitutions described in WO 92/05249, WO 94/25577, WO 95/22615, WO 97/04079 and WO 97/07202.

Parent Lipase Variants

In one aspect, said variant, when compared to said parent, comprises a total of at least three substitutions, said substitutions being selected from one or more of the following groups of substitutions:
A) at least two, or at least three, or at least four, or at least five, or at least six, such as two, three, four, five or six, substitutions in Region I,
b) at least one, at least two, or at least three, or at least four, or at least five, or at least six, such as one, two, three, four, five or six, substitution in Region II,
c) at least one, at least two, or at least three, or at least four, or at least five, or at least six, such as one, two, three, four, five or six, substitution in Region III,
d) and/or at least one, at least two, or at least three, or at least four, or at least five, or at least six, such as one, two, three, four, five or six, substitution in Region IV.

The variant may comprise substitutions, compared to the variant's parent, corresponding to those substitutions listed below in Table 1.

TABLE 1
Some particular variants.

| | | | | Outside |
|---|---|---|---|---|
| Region I | Region II | Region III | Region IV | regions |
| X4V + X227G + | X210K + | X83T + | X58A + X60S | X150G |
| X231R + X233R | X256K | X86V | | |
| X227G + X231R + | X256K | X86V | X58N + X60S | X150G |
| X233R | | | | |
| X231R + X233R | X255Y | | | |
| X231R + X233R | X202G | | | |
| X227G + X231R + | X256K | X86V | | |
| X233R | | | | |
| X4V + X231R + | | | X58N + X60S | |
| X233R | | | | |
| X231R + X233R | | X90R | X58N + X60S | |
| X231R + X233R | X255V | X90A | | |
| X227G + X231R + | X256K | X86V | X58N + X60S | X150G |
| X233R | | | | |
| X231R + X233R | X211L | | X58N + X60S | X147M |
| X231R + X233R | | | | X150K |

In a further particular embodiment the parent lipase is identical to SEQ ID NO:2, and the variants of Table 1 will thus be:

TABLE 2
Some particular variants of SEQ ID NO: 2

| | | | | Outside |
|---|---|---|---|---|
| Region I | Region II | Region III | Region IV | regions |
| Q4V + L227G + | E210K + | S83T + | S58A + V60S | A150G |
| T231R + N233R | P256K | I86V | | |
| L227G + T231R + | P256K | I86V | S58N + V60S | A150G |
| N233R | | | | |
| T231R + N233R | I255Y | | | |
| T231R + N233R | I202G | | | |
| L227G + T231R + | P256K | I86V | | |
| N233R | | | | |
| Q4V + T231R + | | | S58N + V60S | |
| N233R | | | | |
| T231R + N233R | | I90R | S58N + V60S | |
| T231R + N233R | I255V | I90A | | |
| L227G + T231R + | P256K | I86V | S58N + V60S | A150G |
| N233R | | | | |
| T231R + N233R | F211L | | S58N + V60S | L147M |
| T231R + N233R | | | | A150K |

Further substitutions may, e.g., be made according to principles known in the art, e.g. substitutions described in WO 92/05249, WO 94/25577, WO 95/22615, WO 97/04079 and WO 97/07202.
Homology and Alignment

For purposes of the present invention, the degree of homology may be suitably determined by means of computer programs known in the art, such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45), using GAP with the following settings for polypeptide sequence comparison: GAP creation penalty of 3.0 and GAP extension penalty of 0.1.

In the present invention, corresponding (or homologous) positions in the lipase sequences of Absidia reflexa, Absidia corymbefera, Rhizmucor miehei, Rhizopus delemar, Aspergillus niger, Aspergillus tubigensis, Fusarium oxysporum, Fusarium heterosporum, Aspergillus oryzea, Penicilium camembertii, Aspergillus foetidus, Aspergillus niger, Thermomyces lanoginosus(synonym: Humicola lanuginose) and Landerina penisapora are defined by the alignment shown in FIG. 1.

To find the homologous positions in lipase sequences not shown in the alignment, the sequence of interest is aligned to the sequences shown in FIG. 1. The new sequence is aligned to the present alignment in FIG. 1 by using the GAP alignment to the most homologous sequence found by the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). The following settings are used for polypeptide sequence comparison: GAP creation penalty of 3.0 and GAP extension penalty of 0.1.

The parent lipase has a homology of at least 50% with the T. lanuginosus lipase (SEQ ID NO: 2), particularly at least 55%, at least 60%, at least 75%, at least 85%, at least 90%, more than 95% or more than 98%. In a particular embodiment the parent lipase is identical to the T. lanuginosus lipase (SEQ ID NO:2).

—Benefit Risk

The Benefit Risk factor describing the performance compared to the reduced risk for odour smell is defined as: BR=RP_{avg}/R. Lipase variants described herein may have BRs greater than 1, greater than 1.1, or even greater than 1 to about 1000.

—Average Relative Performance

The procedure for calculating average relative performance (RP_{avg}) is found in Example 5 of the present specification. Lipase variants described herein may have (RP_{avg}) of at least 0.8, at least 1.1, at least 1.5, or even at least 2 to about 1000.

—Relative LU/A280

The relative LU/A280 is determined by LU/A280 assay found in Example 4 of the present specification. Lipase variants described herein may have a relative LU/A280 less than 1.00, less than 0.9, less than 0.8 or even from less than 1.00 to about 0.1.

Sources of Polypeptides Having Lipase Activity

A polypeptide of the present invention may be obtained from microorganisms of any genus. For purposes of the present invention, the term “obtained from” as used herein in connection with a given source shall mean that the polypeptide encoded by a nucleotide sequence is produced by the source or by a strain in which the nucleotide sequence from the source has been inserted. In a preferred aspect, the polypeptide obtained from a given source is secreted extracellularly.

A polypeptide of the present invention may be a bacterial polypeptide. For example, the polypeptide may be a gram positive bacterial polypeptide such as a Bacillus polypeptide, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis polypeptide; or a Streptomyces polypeptide, e.g., a Streptomyces lividans or Streptomyces murinus polypeptide; or a gram negative bacterial polypeptide, e.g., an E. coli or a Pseudomonas sp. polypeptide.

A polypeptide of the present invention may also be a fungal polypeptide, and more preferably a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Cryptococcus, Filobasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma polypeptide.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having lipase activity.

In another preferred aspect, the polypeptide is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Aspergillus turbigensis, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Thermomyces lanoginosus(synonym: Humicola lanuginose), Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride polypeptide.

In another preferred aspect, the polypeptide is a Thermomyces polypeptide.

In a more preferred aspect, the polypeptide is a Thermomyces lanuginosus polypeptide, e.g., the polypeptide of SEQ ID NO: 2 with mutations as disclosed in the present application.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of another microorganism. Once a polynucleotide sequence encoding a polypeptide has been detected with the probe(s), the polynucleotide can be isolated or cloned by utilizing techniques which are well known to those of ordinary skill in the art (see, e.g., Sambrook et al., 1989, supra).

Polypeptides of the present invention also include fused polypeptides or cleavable fusion polypeptides in which another polypeptide is fused at the N-terminus or the C-terminus of the polypeptide or fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

Polynucleotides

The lipase of the present invention is preferably derived from a isolated polynucleotides having a nucleotide sequence which encode a polypeptide of the present invention, more preferably nucleotide sequences which encode a polypeptide being a variant of the amino acid sequence of SEQ ID NO: 2 or the mature polypeptide thereof, which differ from the encoding polynucleotide by virtue of the degeneracy of the genetic code. The polypeptide may also be derived from subsequences of SEQ ID NO: 1 which encode fragments of SEQ ID NO: 2 that have lipase activity, said fragments having a BR of at least 1.1 and a RP of at least 0.8.

The techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Thermomyces, or another or related organism and thus, for example, may be an allelic or species variant of the polypeptide encoding region of the nucleotide sequence.

The polypeptide may be derived from polynucleotides having nucleotide sequences which have a degree of identity to the mature polypeptide coding sequence of SEQ ID NO: 1 of at least 60%, preferably at least 65%, more preferably at least 70%, more preferably at least 75%, more preferably at least 80%, more preferably at least 85%, more preferably at least 90%, even more preferably at least 95%, and most preferably at least 97% identity, which encode an active polypeptide having lipase activity and BR of at least 1.1 and RP of at least 0.8.

Modification of a nucleotide sequence encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., artificial variants that differ in specific activity, thermo stability, pH optimum, or the like. The variant sequence may be constructed on the basis of the nucleotide sequence presented as the polypeptide encoding region of SEQ ID NO: 1, e.g., a subsequence thereof, and/or by introduction of nucleotide substitutions which do not give rise to another amino acid sequence of the polypeptide encoded by the nucleotide sequence, but which correspond to the codon usage of the host organism intended for production of the enzyme, or by introduction of nucleotide substitutions which may give rise to a different amino acid sequence.

For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by an isolated polynucleotide of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for lipase activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The polypeptide may be derived from isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) of SEQ ID NO: 1, (ii) the cDNA sequence contained in SEQ ID NO: 1, or (iii) a complementary strand of (i) or (ii); or allelic variants and subsequences thereof (Sambrook et al., 1989, supra), as defined herein.

The polypeptide may be derived from isolated polynucleotides obtained by (a) hybridizing a population of DNA under very low, low, medium, medium-high, high, or very high stringency conditions with (i) nucleotides SEQ ID NO: 1, (ii) the cDNA sequence contained in nucleotides of SEQ ID NO: 1, or (iii) a complementary strand of (i) or (ii); and (b) isolating the hybridizing polynucleotide, which encodes a polypeptide having lipase activity.

Nucleic Acid Constructs

Nucleic acid constructs comprising an isolated polynucleotide of the present invention can be operably linked to one or more control sequences which direct the expression of the coding sequence in a suitable host cell under conditions compatible with the control sequences.

An isolated polynucleotide encoding a polypeptide of the present invention may be manipulated in a variety of ways to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence which is recognized by a host cell for expression of a polynucleotide encoding a polypeptide of the present invention. The promoter sequence contains transcriptional control sequences which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1,ADH2/GAP), Saccharomyces cerevisiae triose phosphate isomerase (TPI), Saccharomyces cerevisiae metallothionine (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

Expression Vectors

Recombinant expression vectors usually comprise a polynucleotide of the present invention, a promoter, and transcriptional and translational stop signals. The various nucleic acids and control sequences described above may be joined together to produce a recombinant expression vector which may include one or more convenient restriction sites to allow for insertion or substitution of the nucleotide sequence encoding the polypeptide at such sites. Alternatively, a nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors preferably contain one or more selectable markers which permit easy selection of transformed cells. A selectable marker is a gene the product of which provides for biocide or viral resistance, resistance to heavy metals, prototrophy to auxotrophs, and the like.

A conditionally essential gene may function as a non-antibiotic selectable marker. Non-limiting examples of bacterial conditionally essential non-antibiotic selectable markers are the dal genes from Bacillus subtilis, Bacillus licheniformis, or other Bacilli, that are only essential when the bacterium is cultivated in the absence of D-alanine. Also the genes encoding enzymes involved in the turnover of UDP-galactose can function as conditionally essential markers in a cell when the cell is grown in the presence of galactose or grown in a medium which gives rise to the presence of galactose. Non-limiting examples of such genes are those from B. subtilis or B. licheniformis encoding UTP-dependent phosphorylase (EC 2.7.7.10), UDP-glucose-dependent uridylyltransferase (EC 2.7.7.12), or UDP-galactose epimerase (EC 5.1.3.2). Also a xylose isomerase gene such as xylA, of Bacilli can be used as selectable markers in cells grown in minimal medium with xylose as sole carbon source. The genes necessary for utilizing gluconate, gntK, and gntP can also be used as selectable markers in cells grown in minimal medium with gluconate as sole carbon source. Other examples of conditionally essential genes are known in the art. Antibiotic selectable markers confer antibiotic resistance to such antibiotics as ampicillin, kanamycin, chloramphenicol, erythromycin, tetracycline, neomycin, hygromycin or methotrexate.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98:61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into the host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

Recombinant host cells usually comprise a polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a unicellular microorganism, e.g., a prokaryote, or a non-unicellular microorganism, e.g., a eukaryote.

Useful unicellular microorganisms are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred aspect, the bacterial host cell is a Bacillus lentus, Bacillus licheniformis, Bacillus stearothermophilus, or Bacillus subtilis cell. In another preferred aspect, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

The host cell may also be a eukaryote, such as a mammalian, insect, plant, or fungal cell.

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, or Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride strain cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Methods of Production

The polypeptide of the present invention can be produced by a method comprising (a) cultivating a cell, which in its wild-type form is capable of producing the polypeptide, under conditions conducive for production of the polypeptide; and (b) recovering the polypeptide. Preferably, the cell is of the genus Aspergillus, and more preferably Aspergillus Oryzae.

Methods for producing a polypeptide of the present invention can also comprise (a) cultivating a host cell under conditions conducive for production of the polypeptide; and (b) recovering the polypeptide.

Methods for producing a polypeptide of the present invention can also comprise (a) cultivating a host cell under conditions conducive for production of the polypeptide, wherein the host cell comprises a mutant nucleotide sequence having at least one mutation in the mature polypeptide coding region of SEQ ID NO: 1, wherein the mutant nucleotide sequence encodes a polypeptide which is a lipase comprised by or comprising the polypeptide of SEQ ID NO: 2, and (b) recovering the polypeptide. In a preferred embodiment the nucleotide sequence encodes a polypeptide which is a lipase comprised by or comprising the mature part of the polypeptide of SEQ ID NO: 2, and (b) recovering the polypeptide.

In the production methods, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

Compositions

Preferably, the compositions are enriched in the polypeptide of the present invention. The term “enriched” indicates that the lipase activity of the composition has been increased, e.g., with an enrichment factor of 1.1.

The composition may comprise a polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be produced, for example, by a microorganism belonging to the genus Aspergillus, preferably Aspergillus aculeatus, Aspergillus awamori, Aspergillus fumigatus, Aspergillus foetidus, Aspergillus japonicus, Aspergillus nidulans, Aspergillus niger, or Aspergillus oryzae; Fusarium, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, preferably Humicola insolens or Humicola lanuginosa; or Trichoderma, preferably Trichoderma harzianum, Trichoderma koningii, Trichoderma longibrachiatum, Trichoderma reesei, or Trichoderma viride.

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Detergent Ingredients

As used herein detergent compositions include articles and cleaning and treatment compositions. As used herein, the term “cleaning and/or treatment composition” includes, unless otherwise indicated, tablet, granular or powder-form all-purpose or “heavy-duty” washing agents, especially laundry detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use. The compositions can also be in unit dose packages, including those known in the art and those that are water soluble, water insoluble and/or water permeable.

The detergent composition of the present invention can comprise one or more lipase variant(s) of the present invention. In addition to the lipase variant(s), the detergent composition will further comprise a detergent ingredient. The non-limiting list of detergent ingredients illustrated hereinafter are suitable for use in the instant compositions and may be desirably incorporated in certain embodiments of the invention, for example to assist or enhance cleaning performance, for treatment of the substrate to be cleaned, or to modify the aesthetics of the cleaning composition as is the case with colorants, dyes or the like. The precise nature of these additional components, and levels of incorporation thereof, will depend on the physical form of the composition and the nature of the cleaning operation for which it is to be used. Suitable detergent ingredients include, but are not limited to, surfactants, builders, chelating agents, dye transfer inhibiting agents, dispersants, enzymes, and enzyme stabilizers, bleach activators, hydrogen peroxide, sources of hydrogen peroxide, preformed peracids, polymeric dispersing agents, brighteners, suds suppressors, dyes, anti-corrosion agents, tarnish inhibitors, perfumes, fabric softeners, carriers, hydrotropes, processing aids, solvents and/or pigments.

Typical detergents would comprise by weight any combination of the following ingredients: 5-30% surfactant, preferably anionic surfactants such as linear alkylbenzenesulfonate and alcohol ethoxysulfate; 0.005-0.1% protease active protein, wherein the protease is preferably selected from Coronase™, FNA, FN4 or Savinase™, 0.001-0.1% amylase active protein, wherein the amylase is preferably selected from Termamyl™ Natalase™, Stainzyme™ and Purastar™ and 0.1-3% chelants, preferably diethylene triamine pentaacetic acid. For granular and tablet products, such typical detergents would additionally comprise by weight: 5-20% bleach, preferably sodium percarbonate; 1-4% bleach activator, preferably TAED and/or 0-30%, preferably 5-30%, more preferably less than 10% builder, such as the aluminosilicate Zeolite A and/or tripolyphosphate.

Bleaching Agents—The detergent compositions of the present invention may comprise one or more bleaching agents.

In general, when a bleaching agent is used, the compositions of the present invention may comprise from about 0.1% to about 50% or even from about 0.1% to about 25% bleaching agent by weight of the subject cleaning composition. Examples of suitable bleaching agents include:

(1) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. soaps; and

(2) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof.

(3) Pre-formed peracids.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

Surfactants—The detergent compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof. When present, surfactant is typically present at a level of from about 0.1% to about 60%, from about 0.1% to about 40%, from about 0.1% to about 12%, from about 1% to about 50% or even from about 5% to about 40% by weight of the subject composition.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

The detergent may optionally contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

Builders—The detergent compositions of the present invention may comprise one or more detergent builders or builder systems. When a builder is used, the subject composition will typically comprise at least about 1%, from about 5% to about 60% or even from about 10% to about 40% builder by weight of the subject composition. Builders include, but are not limited to, the alkali metal, ammonium and alkanolammonium salts of polyphosphates, alkali metal silicates or layered silicates, alkaline earth and alkali metal carbonates, aluminosilicate builders and the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

Chelating Agents—The detergent compositions herein may contain a chelating agent. Suitable chelating agents include copper, iron and/or manganese chelating agents and mixtures thereof. When a chelating agent is used, the subject composition may comprise from about 0.005% to about 15% or even from about 3.0% to about 10% chelating agent by weight of the subject composition.

Brighteners—The detergent compositions of the present invention can also contain additional components that may alter appearance of articles being cleaned, such as fluorescent brighteners. These brighteners absorb in the UV-range and emit in the visible. Suitable fluorescent brightener levels include lower levels of from about 0.01, from about 0.05, from about 0.1 or even from about 0.2 wt % to upper levels of 0.5 or even 0.75 wt %.

Dispersants—The compositions of the present invention can also contain dispersants. Suitable water-soluble organic materials include the homo- or co-polymeric acids or their salts, in which the polycarboxylic acid comprises at least two carboxyl radicals separated from each other by not more than two carbon atoms.

Enzymes—In addition to the lipase variant(s) of the present invention the detergent composition can comprise one or more further enzymes which provide cleaning performance and/or fabric care benefits such as a protease, another lipase, a cutinase, an amylase, a carbohydrase, a cellulase, a pectinase, a mannanase, an arabinase, a galactanase, a xylanase, an oxidase, e.g., a laccase, and/or a peroxidase.

In general the properties of the chosen enzyme(s) should be compatible with the selected detergent, (i.e. pH-optimum, compatibility with other enzymatic and non-enzymatic ingredients, etc.), and the enzyme(s) should be present in effective amounts.

Suitable proteases include those of animal, vegetable or microbial origin. Microbial origin is preferred. Chemically modified or protein engineered mutants are included. The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279), SEQ ID no 4 and SEQ ID no 7 in WO 05/103244. Other suitable serin proteases include those from Micrococcineae spp especially Cellulonas spp and variants thereof as disclosured in WO2005052146. Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 68, 76, 87, 97, 101, 104, 106, 120, 123, 167, 170, 194, 206, 218, 222, 224, 235, 245, 252 and 274, and amongst other variants with the following mutations: (K27R, V104Y, N123S, T124A), (N76D, S103A, V104I), or (S101G, S103A, V104I, G159D, A232V, Q236H, Q245R, N248D, N252K). Other examples of useful proteases are the variants described in WO 05/052146 especially the variants with substitutions in one or more of the following positions: 14, 16, 35, 65, 75, 76, 79, 123, 127, 159 and 179

Preferred commercially available protease enzymes include Alcalase™, Savinase™, Primase™, Duralase™, Esperase™, Coronase™, Polarzyme™ and Kannase™ (Novozymes A/S), Maxatase™, Maxacal™, Maxapem™, Properase™, Purafect™, Purafect Prime™, Purafect OxP™, FNA, FN2, FN3 and FN4 (Genencor International Inc.).

Lipases include those of bacterial or fungal origin. Chemically modified or protein engineered mutants are included. Examples of useful lipases include lipases from Humicola (synonym Thermomyces), e.g. from H. lanuginosa (synonymous T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Other commercially available lipase enzymes include Lipolase™, Lipolase Ultra™ and Lipex™ (Novozymes A/S).

Suitable amylases (αand/or β) include those of bacterial or fungal origin. Chemically modified or protein engineered mutants are included. Amylases include, for example, α-amylases obtained from Bacillus, e.g. a special strain of B. licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramyl™, Termamyl™, Stainzyme™, Stainzyme Ultra™, Fungamyl™ and BAN™ (Novozymes A/S), Rapidase™ and Purastar™ (from Genencor International Inc.).

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Renozyme™, Celluclean™, Endolase™, Celluzyme™, and Carezyme™ (Novozymes A/S), Clazinase™, and Puradax HA™ (Genencor International Inc.), and KAC-500(B)™ (Kao Corporation).

Peroxidases/Oxidases:

Suitable peroxidases/oxidases include those of plant, bacterial or fungal origin. Chemically modified or protein engineered mutants are included. Examples of useful peroxidases include peroxidases from Coprinus, e.g. from C. cinereus, and variants thereof as those described in WO 93/24618, WO 95/10602, and WO 98/15257.

Commercially available peroxidases include Guardzyme™ (Novozymes A/S).

When present in a cleaning composition, the aforementioned enzymes may be present at levels from about 0.00001% to about 2%, from about 0.0001% to about 1% or even from about 0.001% to about 0.5% enzyme protein by weight of the composition.

Enzyme Stabilizers—Enzymes for use in detergents can be stabilized by various techniques. The enzymes employed herein can be stabilized by the presence of water-soluble sources of calcium and/or magnesium ions in the finished compositions that provide such ions to the enzymes. Further conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, may also be used and the composition may be formulated as described in e.g. WO 92/19709 and WO 92/19708.

Solvents—Suitable solvents include water and other solvents such as lipophilic fluids.

Examples of suitable lipophilic fluids include siloxanes, other silicones, hydrocarbons, glycol ethers, glycerine derivatives such as glycerine ethers, perfluorinated amines, perfluorinated and hydrofluoroether solvents, low-volatility nonfluorinated organic solvents, diol solvents, other environmentally-friendly solvents and mixtures thereof.

Washing Method

The present invention includes a method for cleaning and/or treating a situs inter alia a surface or fabric. Such method includes the steps of contacting an embodiment of Applicants' cleaning composition, in neat form or diluted in a wash liquor, with at least a portion of a surface or fabric then optionally rinsing such surface or fabric. The surface or fabric may be subjected to a washing step prior to the aforementioned rinsing step. For purposes of the present invention, washing includes but is not limited to, scrubbing, and mechanical agitation. As will be appreciated by one skilled in the art, the cleaning compositions of the present invention are ideally suited for use in laundry applications. Accordingly, the present invention includes a method for laundering a fabric. The method comprises the steps of contacting a fabric to be laundered with a said cleaning laundry solution comprising at least one embodiment of Applicants' cleaning composition, cleaning additive or mixture thereof. The fabric may comprise most any fabric capable of being laundered in normal consumer use conditions. The solution preferably has a pH of from about 8 to about 10.5. The compositions may be employed at concentrations of from about 100 ppm, preferably 500 ppm to about 15,000 ppm in solution. The water temperatures typically range from about 5° C. to about 90° C. The invention may be particularly beneficial at low water temperatures such as below 30° C. or below 25 or 20° C. The water to fabric ratio is typically from about 1:1 to about 30:1.

### LIPASE VARIANTS EXAMPLES

Chemicals used as buffers and substrates are commercial products of at least reagent grade.
Media and Solutions: LAS (Surfac PS™) and Zeolite A (Wessalith P™). Other ingredients used are standard laboratory reagents.
Materials: EMPA221 from EMPA St. Gallen, Lerchfeldstrasse 5, CH-9014 St. Gallen, Switzerland

### Example 1

### Production of Enzyme

A plasmid containing the gene encoding the lipase is constructed and transformed into a suitable host cell using standard methods of the art.

Fermentation is carried out as a fed-batch fermentation using a constant medium temperature of 34° C. and a start volume of 1.2 liter. The initial pH of the medium is set to 6.5. Once the pH has increased to 7.0 this value is maintained through addition of 10% H3PO4. The level of dissolved oxygen in the medium is controlled by varying the agitation rate and using a fixed aeration rate of 1.0 liter air per liter medium per minute. The feed addition rate is maintained at a constant level during the entire fed-batch phase.

The batch medium contains maltose syrup as carbon source, urea and yeast extract as nitrogen source and a mixture of trace metals and salts. The feed added continuously during the fed-batch phase contains maltose syrup as carbon source whereas yeast extract and urea is added in order to assure a sufficient supply of nitrogen.

Purification of the lipase may be done by use of standard methods known in the art, e.g. by filtering the fermentation supernatant and subsequent hydrophobic chromatography and anion exchange, e.g. as described in EP 0 851 913 EP, Example 3.

### Example 2

### AMSA—Automated Mechanical Stress Assay—for Calculation of RP

The enzyme variants of the present application are tested using the Automatic Mechanical Stress Assay (AMSA). With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress. For further description see WO 02/42740 especially the paragraph “Special method embodiments” at page 23-24. The containers, which contain the detergent test solution, consist of cylindrical holes (6 mm diam, 10 mm depth) in a metal plate. The stained fabric (test material) lies on the top of the metal plate and is used as a lid and seal on the containers. Another metal plate lies on the top of the stained fabric to avoid any spillage from each container. The two metal plates together with the stained fabric are vibrated up and down at a frequency of 30 Hz with an amplitude of 2 mm.

The assay is conducted under the experimental conditions specified below:

TABLE 3

| | |
|---|---|
| Test solution | 0.5 g/l LAS |
| | 0.52 g/l Na2CO3 |
| | 1.07 g/l Zeolite A |
| | 0.52 g/l Trisodium citrate |
| Test solution volume | 160 micro 1 |
| pH | As is (≈9.9) |
| Wash time | 20 minutes |
| Temperature | 30° C. |
| Water hardness | 15°dH |
| | Ratio of Ca²⁺/Mg²⁺/NaHCO₃: 4:1:7.5 |
| Enzyme concentration in | 0.125, 0.25, 0.50, 1.0 mg enzyme |
| test solution | protein/liter (mg ep/1) |
| Drying | Wash performance: After washing the |
| | textile pieces are immediately flushed in tap |
| | water and air-dried at 85 C. in 5 mm |
| | Odour: After washing the textile pieces are |
| | immediately flushed in tap water and dried |
| | at room temperature (20° C.) for 2 hours |
| Test material | Cream turmeric swatch as described below |
| | (EMPA221 used as cotton textile) |

Cream-turmeric swatches are prepared by mixing 5 g of turmeric (Santa Maria, Denmark) with 100 g cream (38% fat, Arla, Denmark) at 50° C., the mixture is left at this temperature for about 20 minutes and filtered (50° C.) to remove any un-dissolved particles. The mixture is cooled to 20° C. and woven cotton swatches, EMPA221, are immersed in the cream-turmeric mixture and afterwards allowed to dry at room temperature over night and frozen until use. The preparation of cream-tumeric swatches is disclosed in the patent application PA 2005 00775, filed 27 May 2005.

The performance of the enzyme variant is measured as the brightness of the colour of the textile samples washed with that specific enzyme variant. Brightness can also be expressed as the intensity of the light reflected from the textile sample when luminated with white light. When the textile is stained the intensity of the reflected light is lower, than that of a clean textile. Therefore the intensity of the reflected light can be used to measure wash performance of an enzyme variant.

Color measurements are made with a professional flatbed scanner (PFU DL2400pro), which is used to capture an image of the washed textile samples. The scans are made with a resolution of 200 dpi and with an output color depth of 24 bits. In order to get accurate results, the scanner is frequently calibrated with a Kodak reflective IT8 target.

To extract a value for the light intensity from the scanned images, a special designed software application is used (Novozymes Color Vector Analyzer). The program retrieves the 24 bit pixel values from the image and converts them into values for red, green and blue (RGB). The intensity value (Int) is calculated by adding the RGB values together as vectors and then taking the length of the resulting vector:
Int=\sqrt{r²+g²+b²}.

The wash performance (P) of the variants is calculated in accordance with the below formula: P=Int(v)−Int(r) where

Int(v) is the light intensity value of textile surface washed with the tested enzyme and

Int(r) is the light intensity value of textile surface washed without the tested enzyme.

A relative performance score is given as the result of the AMSA wash in accordance with the definition: Relative Performance scores (RP) are summing up the performances (P) of the tested enzyme variants against the reference enzyme:
RP=P(test enzyme)/P(reference enzyme)
RPavg indicates the average relative performance compared to the reference enzyme at all four enzyme concentrations (0.125, 0.25, 0.5, 1.0 mg ep/l)
RPavg=avg(RP(0.125),RP(0.25)RP(0.5),RP(1.0))

A variant is considered to exhibit improved wash performance, if it performs better than the reference.

In the context of the present invention the reference enzyme is the lipase of SEQ ID NO:2 with the substitutions T231R+N233R

### Example 3

### GC—Gas Chromatograph—for Calculation of Risk Factor

The butyric acid release from the lipase washed swatches are measured by Solid Phase Micro Extraction Gas Chromatography (SPME-GC) using the following method. Four textile pieces (5 mm in diameter), washed in the specified solution in Table 1 containing 1 mg/L lipase, are transferred to a Gas Chromatograph (GC) vial. The samples are analysed on a Varian 3800 GC equipped with a Stabilwax-DA w/Integra-Guard column (30 m, 0.32 mm ID and 0.25 micro-m df) and a Carboxen PDMS SPME fibre (75 micro-m). Each sample is preincubated for 10 min at 40° C. followed by 20 min sampling with the SPME fibre in the head-space over the textile pieces. The sample is subsequently injected onto the column (injector temperature=250° C.). Column flow=2 ml Helium/min. Column oven temperature gradient: 0 min=40° C., 2 min=40° C., 22 min=240° C., 32 min=240° C. The butyric acid is detected by FID detection and the amount of butyric acid is calculated based on a butyric acid standard curve.

The Risk Performance Odour, R, of a lipase variant is the ratio between the amount of released butyric acid from the lipase variant washed swatch and the amount of released butyric acid from a swatch washed with the mature part of the lipase of SEQ ID NO: 2, after both values have been corrected for the amount of released butyric acid from a non-lipase washed swatch. The risk (R) of the variants is calculated in accordance with the below formula:
Odour=measured in μg butyric acid developed at 1 mg enzyme protein/1 corrected for blank
α_{test enzyme}=Odour_{test enzyme}−Blank
α_{reference enzyme}=Odour_{reference enzyme}−Blank
R=α_{test enzyme}/α_{reference enzyme}
A variant is considered to exhibit reduced odor compared to the reference, if the R factor is lower than 1.

### Example 4

### Activity (LU) Relative to Absorbance at 280 nm

The activity of a lipase relative to the absorbance at 280 nm is determined by the following assay LU/A280:

A substrate for lipase is prepared by emulsifying tributyrin (glycerin tributyrate) using gum Arabic as emulsifier. The hydrolysis of tributyrin at 30° C. at pH 7 or 9 is followed in a pH-stat titration experiment. One unit of lipase activity (1 LU) equals the amount of enzyme capable of releasing 1 micro mol butyric acid/min at pH 7.

The absorbance of the purified lipase at 280 nm is measured (A280) and the ratio LU/A280 is calculated. The relative LU/A280 is calculated as the LU/A280 of the variant divided by the LU/A280 of a reference enzyme. In the context of the present invention the reference enzyme is the mature part of SEQ ID NO:2 with the mutations T231R and N233R.

### Example 5

### BR—Benefit Risk

The Benefit Risk factor describing the performance compared to the reduced risk for odour smell is thus defined as: BR=RP_{avg}/R

A variant is considered to exhibit improved wash performance and reduced odor, if the BR factor is higher than 1.

Applying the above methods the following results are obtained:

TABLE 4

| | Mutations in mature part of polypeptide | | | |
|---|---|---|---|---|
| Variant | of SEQ ID NO: 2 | RP | BR | LU/A280 |
| 1 | I202G + T231R + N233R | 0.84 | 1.41 | not |
| | | | | determined |
| 2 | I86V + L227G + T231R + N233R + | 1.08 | 1.52 | 1700 |
| | P256K | | | |
| 3 | Q4V + S58N + V60S + T231R + N233R | 0.87 | 1.73 | 1950 |
| 4 | S58N + V60S + 190R + T231R, N233R | 1.06 | 1.27 | 2250 |
| 5 | I255Y + T231R + N233R | 1.19 | 1.17 | 3600 |
| 6 | I90A + T231R + N233R + I255V | 1.13 | 1.14 | 2700 |
| Reference | T231R + N233R | 1.00 | 1.00 | 3650 |
| 7 | G91A + E99K + T231R + N233R + | 0.43 | not | 850 |
| | Q249R + 270H + 271T + 272P + 273S + | | determined | |
| | 274S + 275G + 276R + 277G + 278G + | | | |
| | 279H + 280R | | | |
| 8 | G91A + E99K + T231R, N233R + | 0.13 | not | 500 |
| | Q249R + 270H + 271T + 272P + 273S + | | determined | |
| | 274S + 275G + 276R + 277G + 278G | | | |

The reference lipase and variants 7 and 8 in Table 4 are described in WO 2000/060063.

### Example 6

### BR—Benefit Risk

The Benefit Risk was measured for the variants listed in Table 5. The Benefit Risk factor was measured in the same way as described in Example 5 and it was found to be above 1 for all the listed variants.

TABLE 5

| Variant | Mutations in SEQ ID NO: 2 |
|---|---|
| Reference | T231R + N233R |
| 9 | L97V + T231R + N233R |
| 10 | A150G + T231R + N233R |
| 11 | I90R + T231R + N233R |
| 12 | I202V + T231R + N233R |
| 13 | L227G + T231R + N233R + P256K |
| 14 | I90A + T231R + N233R |
| 15 | T231R + N233R + I255P |
| 16 | I90V + I255V + T231R + N233R |
| 17 | F211L + L227G + T231R + N233R + I255L + P256K |
| 18 | S58N + V60S + T231R + N233R + Q249L |
| 19 | S58N + V60S + T231R + N233R + Q249I |
| 20 | A150G + L227G + T231R + N233R + P256K |
| 21 | K46L + S58N + V60S + T231R + N233R + Q249L + D254I |
| 22 | Q4L + E43T + K46I + S58N + V60S + T231R + N233R + Q249L + D254I |
| 23 | Q4L + S58N + V60S + T231R + N233R + Q249L + D254I |
| 24 | K46I + S58N + V60S + T231R + N233R + Q249L + D254L |
| 25 | K46L + S58N + V60S + K223I + T231R + N233R + D254I |
| 26 | E43T + K46I + S58N + V60S + T231R + N233R + Q249L + D254I |
| 27 | S58N + V60S + I86V + A150G + L227G + T231R + N233R + P256K |
| 28 | K24R + K46R + K74R + I86V + K98R + K127R + D137K + A150G + K223R + T231R + N233R |
| 29 | S58A + V60A + I86V + T231R + N233R |
| 30 | K24R + K46R + S58N + V60S + K74R + I86V + K98R + K127R + D137K + K223R + T231R + N233R |
| 31 | S58A + V60A + I86V + A150G + T231R + N233R |
| 32 | S58N + V60V + D62G + T231R + N233R |
| 33 | Q4V + S58N + V60S + I86V + T231R + N233R + Q249L |
| 34 | Q4V + S58N + V60S + I86V + A150G + T231R + N233R + I255V |
| 35 | Q4V + S58N + V60S + I90A + A150G + T231R + N233R + I255V |
| 36 | Y53A + S58N + V60S + T231R + N233R + P256L |
| 37 | I202L + T231R + N233R + I255A |
| 38 | S58A + V60S + I86V + A150G + L227G + T231R + N233R + P256K |
| 39 | D27R + S58N + V60S + I86V + A150G + L227G + T231R + N233R + P256K |
| 40 | V60K + I86V + A150G + L227G + T231R + N233R + P256K |
| 41 | Q4V + S58A + V60S + S83T + I86V + A150G + E210K + L227G + T231R + N233R + P256K |
| 42 | Q4V + V60K + S83T + I86V + A150G + L227G + T231R + N233R + P256K |
| 43 | D27R + V60K + I86V + A150G + L227G + T231R + N233R + P256K |
| 44 | Q4N + L6S + S58N + V60S + I86V + A150G + L227G + T231R + N233R + P256K |
| 45 | E1N + V60K + I86V + A150G + L227G + T231R + N233R + P256K |
| 46 | V60K + I86V + A150G + K223N + G225S + T231R + N233R + P256K |
| 47 | E210V + T231R + N233R + Q249R |
| 48 | S58N + V60S + E210V + T231R + N233R + Q249R |
| 49 | Q4V + V60K + I90R + T231R + N233R + I255V |
| 50 | Q4V + V60K + A150G + T231R + N233R |
| 51 | V60K + S83T + T231R + N233R |
| 52 | V60K + A150G + T231R + N233R + I255V |
| 53 | T231R + N233G + D234G |
| 54 | S58N + V60S + I86V + A150G + E210K + L227G + T231R + N233R + Q249R + P256K |
| 55 | S58N + V60S + I86V + A150G + E210K + L227G + T231R + N233R + I255A + P256K |
| 56 | S58N + V60S + I86V + A150G + G156R + E210K + L227G + T231R + N233R + I255A + P256K |
| 57 | S58T + V60K + I86V + N94K + A150G + E210V + L227G + T231R + N233R + P256K |
| 58 | S58T + V60K + I86V + D102A + A150G + L227G + T231R + N233R + P256K |
| 59 | S58T + V60K + I86V + D102A + A150G + E210V + L227G + T231R + N233R + P256K |
| 60 | S58T + V60K + S83T + I86V + N94K + A150G + E210V + L227G + T231R + N233R + P256K |
| 61 | S58A + V60S + I86V + T143S + A150G + L227G + T231R + N233R + P256K |
| 62 | G91S + D96V + D254R |
| 63 | V60L + G91M + T231W + Q249L |
| 64 | T37A + D96A + T231R + N233R + Q249G |
| 65 | E56G + E87D + T231R + N233R + D254A |
| 66 | E210K + T231R + N233R |
| 67 | D27H + E87Q + D96N + T231R + N233R + D254V |
| 68 | F181L + E210V + T231R + N233R |
| 69 | D27N + D96G + T231R + N233R |
| 70 | D96N + T231R + N233R |
| 71 | T231R + N233I + D234G |
| 72 | S58K + V60L + E210V + Q249R |
| 73 | S58H + V60L + E210V + Q249R |
| 74 | Q4V + F55V + I86V + T231R + N233R + I255V |
| 75 | Q4V + S58T + V60K + T199L + N200A + E210K + T231R + N233R + I255A + P256K |
| 76 | Q4V + D27N + V60K + T231R + N233R |
| 77 | I90F + I202P + T231R + N233R + I255L |
| 78 | S58N + V60S + D158N + T231R + N233R |
| 79 | S58N + V60S + S115K + T231R + N233R |
| 80 | S58N + V60S + L147M + A150G + F211L + T231R + N233R |
| 81 | V60K + A150G + T231R + N233R |
| 82 | I90V + L227G + T231R + N233R + P256K |
| 83 | T231R + N233R + I255S |
| 84 | I86G + T231R + N233R |
| 85 | V60K + I202V + E210K + T231R + N233R + I255A + P256K |
| 86 | I90G + I202L + T231R + N233R + I255S |
| 87 | S58G + V60G + T231R + N233R |

The reference lipase is described in WO 2000/060063.

### DETERGENT EXAMPLES

Abbreviated component identifications for the examples are as follows:

| | |
|---|---|
| LAS | Sodium linear C₁₁₋₁₃ alkyl benzene sulphonate. |
| CxyAS | Sodium C₁ₓ-C_{1y} alkyl sulfate. |
| CxyEzS | C₁ₓ-C_{1y} sodium alkyl sulfate condensed with an average of z |
| | moles of ethylene oxide. |
| CxyEy | C₁ₓ-C_{1y} alcohol with an average of ethoxylation of z |
| QAS | R₂•N + (CH₃)₂(C₂H₄OH) with R2 = C₁₀-C₁₂ |
| Silicate | Amorphous Sodium Silicate (SiO₂:Na₂O ratio = 1.6-3.2:1). |
| Zeolite A | Hydrated Sodium Aluminosilicate of formula Na₁₂(A1O₂SiO₂)₁₂•27H₂O |
| | having a primary particle size in the range from 0.1 to 10 |
| | micrometers (Weight expressed on an anhydrous basis). |
| (Na−)SKS-6 | Crystalline layered silicate of formula δ-Na₂Si₂O₅. |
| Citrate | Tri-sodium citrate dihydrate. |
| Citric | Anhydrous citric acid. |
| Carbonate | Anhydrous sodium carbonate. |
| Sulphate | Anhydrous sodium sulphate. |
| MA/AA | Random copolymer of 4:1 acrylate/maleate, average molecular |
| | weight about 70,000-80,000. |
| AA polymer | Sodium polyacrylate polymer of average molecular weight 4,500. |
| PB1/PB4 | Anhydrous sodium perborate monohydrate/tetrahydrate. |
| PC3 | Anhydrous sodium percarbonate [2.74 Na₂CO₃•3H₂O₂] |
| TAED | Tetraacetyl ethylene diamine. |
| NOBS | Nonanoyloxybenzene sulfonate in the form of the sodium salt. |
| DTPA | Diethylene triamine pentaacetic acid. |
| HEDP | Hydroxyethane di phosphonate |
| EDDS | Na salt of Ethylenediamine-N,N′-disuccinic acid, (S,S) isomer |
| STPP | Sodium tripolyphosphate |
| Protease | Proteolytic enzyme sold under the tradename Savinase ®, |
| | Alcalase ®, Everlase ®, Coronase ®, Polarzyme ®, by Novozymes |
| | A/S, Properase ®, Purafect ®, Purafect MA ® and Purafect Ox ® |
| | sold by Genencor and proteases described in patents WO 91/06637 |
| | and/or WO 95/10591 and/or EP 0 251 446 such as FNA, FN3 |
| | and/or FN4. |
| Amylase | Amylolytic enzyme sold under the tradename Purastar ®, Purafect |
| | Oxam ® sold by Genencor; Termamyl ®, Fungamyl ® Duramyl ®, |
| | Stainzyme ® and Natalase ® sold by Novozymes A/S. |
| Lipase | Any lipase variant 1 to 5 described in example 5 table 2, and |
| | combinations thereof. |
| Mannanase | Mannaway ® sold by Novozymes |
| CMC or HEC | Carboxymethyl or Hydroxyethyl or ester modified cellulose. |
| or EMC | |
| SS Agglom. | Suds Suppressor agglomerate: 12% Silicone/silica, 18% stearyl |
| | alcohol, 70% starch in granular form. |
| TEPAE | Tetreaethylenepentaamine ethoxylate. |
| pH | Measured as a 1% solution in distilled water at 20° C. |

### Example A

Bleaching detergent compositions having the form of granular laundry detergents are exemplified by the following formulations.

| | A | B | C | D | E | F |
|---|---|---|---|---|---|---|
| LAS | 20 | 22 | 20 | 15 | 20 | 20 |
| QAS | 0.7 | 1 | 1 | 0.6 | 0.0 | 0.7 |
| C25E3S | 0.9 | 0.0 | 0.9 | 0.0 | 0.0 | 0.9 |
| C25E7 | 0.0 | 0.5 | 0.0 | 1 | 3 | 1 |
| STPP | 23 | 30 | 23 | 17 | 12 | 23 |
| Zeolite A | 0.0 | 0.0 | 0.0 | 0.0 | 10 | 0.0 |
| Silicate | 7 | 7 | 7 | 7 | 7 | 7 |
| Carbonate | 15 | 14 | 15 | 18 | 15 | 15 |
| AA Polymer | 1 | 0.0 | 1 | 1 | 1.5 | 1 |
| CMC | 1 | 1 | 1 | 1 | 1 | 1 |
| Protease | 0.1 | 0.07 | 0.1 | 0.1 | 0.1 | 0.1 |
| 32.89 mg/g | | | | | | |
| Amylase | 0.1 | 0.1 | 0.1 | 0.0 | 0.1 | 0.1 |
| 8.65 mg/g | | | | | | |
| Lipase 18 mg/g | 0.03 | 0.07 | 0.3 | 0.1 | 0.07 | 0.1 |
| Brightener - | 0.06 | 0.0 | 0.06 | 0.18 | 0.06 | 0.06 |
| Tinopal | | | | | | |
| AMS (Ciba) | | | | | | |
| Brightener - | 0.1 | 0.06 | 0.1 | 0.0 | 0.1 | 0.1 |
| Tinopal | | | | | | |
| CBS-X (Ciba) | | | | | | |
| DTPA | 0.6 | 0.3 | 0.6 | 0.25 | 0.6 | 0.6 |
| MgSO₄ | 1 | 1 | 1 | 0.5 | 1 | 1 |
| PC3 | 0.0 | 5.2 | 0.1 | 0.0 | 0.0 | 0.0 |
| PB1 | 4.4 | 0.0 | 3.85 | 2.09 | 0.78 | 3.63 |
| NOBS | 1.9 | 0.0 | 1.66 | 1.77 | 0.33 | 0.75 |
| TAED | 0.58 | 1.2 | 0.51 | 0.0 | 0.015 | 0.28 |
| Sulphate/ | Balance | Balance | Balance | Balance | Balance | Balance |
| Moisture | to 100% | to 100% | to 100% | to 100% | to 100% | to 100% |

Any of the compositions in Example A is used to launder fabrics at a concentration of 600-10000 ppm in water, with typical median conditions of 2500 ppm, 25^{O} C, and a 25:1 water:cloth ratio. The typical pH is about 10 but can be can be adjusted by altering the proportion of acid to Na-salt form of alkylbenzenesulfonate.

### Example B

Bleaching detergent compositions having the form of granular laundry detergents are exemplified by the following formulations.

| | A | B | C | D |
|---|---|---|---|---|
| LAS | 8 | 7.1 | 7 | 6.5 |
| C25E3S | 0 | 4.8 | 0 | 5.2 |
| C68S | 1 | 0 | 1 | 0 |
| C25E7 | 2.2 | 0 | 3.2 | 0 |
| QAS | 0.75 | 0.94 | 0.98 | 0.98 |
| (Na−)SKS-6 | 4.1 | 0 | 4.8 | 0 |
| Zeolite A | 20 | 0 | 17 | 0 |
| Citric | 3 | 5 | 3 | 4 |
| Carbonate | 15 | 20 | 14 | 20 |
| Silicate | 0.08 | 0 | 0.11 | 0 |
| Soil release agent | 0.75 | 0.72 | 0.71 | 0.72 |
| MA/AA | 1.1 | 3.7 | 1.0 | 3.7 |
| CMC | 0.15 | 1.4 | 0.2 | 1.4 |
| Protease (56.00 mg active/g) | 0.37 | 0.4 | 0.4 | 0.4 |
| Termamyl (21.55 mg active/g) | 0.3 | 0.3 | 0.3 | 0.3 |
| Lipase (18.00 mg active/g) | 0.05 | 0.15 | 0.1 | 0.5 |
| Amylase (8.65 mg active/g) | 0.1 | 0.14 | 0.14 | 0.3 |
| TAED | 3.6 | 4.0 | 3.6 | 4.0 |
| PC3 | 13 | 13.2 | 13 | 13.2 |
| EDDS | 0.2 | 0.2 | 0.2 | 0.2 |
| HEDP | 0.2 | 0.2 | 0.2 | 0.2 |
| MgSO₄ | 0.42 | 0.42 | 0.42 | 0.42 |
| Perfume | 0.5 | 0.6 | 0.5 | 0.6 |
| SS Agglom. | 0.05 | 0.1 | 0.05 | 0.1 |
| Soap | 0.45 | 0.45 | 0.45 | 0.45 |
| Sulphate | 22 | 33 | 24 | 30 |

| | |
|---|---|
| Water & Miscellaneous | Balance to 100% |

Any of the above compositions in Example B is used to launder fabrics at a concentration of 10,000 ppm in water, 20-90^{O} C, and a 5:1 water:cloth ratio. The typical pH is about 10 but can be can be adjusted by altering the proportion of acid to Na-salt form of alkylbenzenesulfonate.

### Example C

| | A | B | C | D | E | F |
|---|---|---|---|---|---|---|
| | (wt %) | (wt %) | (wt %) | (wt %) | (wt %) | (wt %) |
| C25E1.8S | 11 | 10 | 4 | 6.32 | 6.0 | 8.2 |
| LAS | 4 | 5.1 | 8 | 3.3 | 4.0 | 3.0 |
| Sodium | 1.6 | 0.09 | 1.2 | 0.04 | 1.6 | 1.2 |
| formate | | | | | | |
| Sodium | 2.3 | 3.8 | 1.7 | 1.9 | 2.3 | 1.7 |
| hydroxide | | | | | | |
| Monoethanol- | 1.4 | 1.490 | 1.0 | 0.7 | 1.35 | 1.0 |
| amine | | | | | | |
| Diethylene | 5.5 | 0.0 | 4.1 | 0.0 | 5.500 | 4.1 |
| glycol | | | | | | |
| C23E9 | 0.4 | 0.6 | 0.3 | 0.3 | 2 | 0.3 |
| DTPA | 0.15 | 0.15 | 0.11 | 0.07 | 0.15 | 0.11 |
| Citric Acid | 2.5 | 3.96 | 1.88 | 1.98 | 2.5 | 1.88 |
| C₁₂₋₁₄ dimethyl | 0.3 | 0.73 | 0.23 | 0.37 | 0.3 | 0.225 |
| Amine Oxide | | | | | | |
| C₁₂₋₁₈ | 0.8 | 1.9 | 0.6 | 0.99 | 0.8 | 0.6 |
| Fatty Acid | | | | | | |
| Borax | 1.43 | 1.5 | 1.1 | 0.75 | 1.43 | 1.07 |
| Ethanol | 1.54 | 1.77 | 1.15 | 0.89 | 1.54 | 1.15 |
| TEPAE¹ | 0.3 | 0.33 | 0.23 | 0.17 | 0.0 | 0.0 |
| ethoxylated | 0.8 | 0.81 | 0.6 | 0.4 | 0.0 | 0.0 |
| hexamethylene | | | | | | |
| diamine² | | | | | | |
| 1,2- | 0.0 | 6.6 | 0.0 | 3.3 | 0.0 | 0.0 |
| Propanediol | | | | | | |
| Protease* | 36.4 | 36.4 | 27.3 | 18.2 | 36.4 | 27.3 |
| Mannanase* | 1.1 | 1.1 | 0.8 | 0.6 | 1.1 | 0.8 |
| Amylase* | 7.3 | 7.3 | 5.5 | 3.7 | 7.3 | 5.5 |
| Lipase* | 10 | 3.2 | 0.5 | 3.2 | 2.4 | 3.2 |
| Water, | Balance | Balance | Balance | Balance | Balance | Balance |
| perfume, | | | | | | |
| dyes & others | | | | | | |

*Numbers quoted in mg enzyme/100 g
¹as described in U.S. Pat. No. 4,597,898.
²available under the tradename LUTENSIT ® from BASF and such as those described in WO 01/05874

All documents cited in the Detailed Description of the Invention are in relevant part incorporated herein by reference: the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A detergent composition comprising a detergent ingredient and a polypeptide having lipase activity and which further has a Average Relative Performance of at least 0.8 and a Benefit Risk of at least 1.1 at the test conditions given in the specification.

2. A detergent composition according to claim 1 wherein the polypeptide has a relative LU/A280 less than 1.00 at the test conditions given in the specification.

3. A detergent composition according to claim 1 wherein the polypeptide is a bacterial polypeptide.

4. A detergent composition according to claim 1 wherein the polypeptide is a fungal polypeptide.

5. A detergent composition according to claim 4 wherein the polypeptide is a Thermomyces polypeptide.

6. A detergent composition according to claim 5 wherein the polypeptide of is a Thermomyces lanuginosus polypeptide.

7. A detergent composition according to claim 1 wherein the polypeptide is a variant of a lipase comprised by the polypeptide of SEQ ID NO: 2.

8. A detergent composition according to claim 1 wherein the polypeptide is a variant of a lipase comprised by the mature part of the polypeptide of SEQ ID NO: 2.

9. A detergent composition according to claim 1 wherein the polypeptide is a variant of a lipase comprising the polypeptide of SEQ ID NO: 2.

10. A detergent composition according to claim 1 wherein the polypeptide is a variant of a lipase comprising the mature part of the polypeptide of SEQ ID NO: 2.

11. A detergent composition according to claim 1 wherein the polypeptide is encoded by a polynucleotide which hybridizes under at least high stringency conditions with nucleotides 644 to 732 of SEQ ID NO: 1 or a complementary strand hereto.

12. A detergent composition according to claim 1 wherein the detergent ingredient is 0.1 to 40% anionic surfactant, preferably from 0.1 to 12%.

13. A detergent composition according to claim 12 wherein the anionic surfactant is an alkoxylated alkyl sulphate.

14. A detergent composition according to claim 1 wherein the detergent ingredient is 5 to 30% aluminosilicate and/or phosphate builder.

15. A detergent composition according to claim 1 wherein the detergent ingredient is a source of peroxide and a bleach activator.

16. A detergent according to claim 1 wherein said detergent is a liquid detergent composition or a solid detergent composition.

17. A detergent according to claim 16 wherein said detergent is a granular detergent composition.

18. A detergent according to claim 1 wherein said detergent is a solid tablet or a liquid encapsulated in a soluble film unit dose composition.

19. A washing process comprising laundering textile articles in an aqueous solution comprising the detergent composition according to claim 1.

20. A washing process according to claim 19 comprising the steps of:
(a) optionally pretreating the soils and stains with the compositions of claim 1 to form an optionally pretreated surface;
(b) adding an effective amount of the compositions of claim 1 to water to form from an aqueous washing solution comprising about 500 to about 10000 ppm of the composition;
(c) contacting the aqueous washing solution with the optionally pretreated surface, and
(d) optionally providing agitation to the aqueous washing solution and the optionally pretreated surface.

21. A washing process according to claims 19 in which the aqueous solution is at a temperature below 30° C.


SEQUENCE LISTING (4 sequences)

SEQ ID NO: 1 - DNA, 873 bp, Thermomyces lanuginosus
  CDS (1)..(873)
  sig_peptide (1)..(51)
  propep (52)..(66)
  mat_peptide (67)..()
atgaggagctcccttgtgctgttctttgtctctgcgtggacggccttggccagtcctattcgtcgagaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctt

SEQ ID NO: 2 - protein, 291 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 3 - protein, 12 aa, Artificial
  Sequence used for aligment example
ACMSHTWGERNL

SEQ ID NO: 4 - protein, 14 aa, Artificial
  Sequence used for alignment example
HGWGEDANLAMNPS